(19) United States
(12) Patent Application Publication
(10) Pub. No.: US 2014/0336355 A1
(43) Pub. Date: Nov. 13, 2014

(54) ION-EXCHANGER MATERIAL WITH HIGH SALT-TOLERANCE

(71) Applicants: Markus Arendt, Hockenheim (DE); Gerhard Stumm, Hamburg (DE); Martin Welter, Heidelberg (DE); Thomas Schwarz, Leichlingen (DE)

(72) Inventors: Markus Arendt, Hockenheim (DE); Gerhard Stumm, Hamburg (DE); Martin Welter, Heidelberg (DE); Thomas Schwarz, Leichlingen (DE)

(73) Assignee: INSTRACTION GMBH, Mannheim (DE)

(21) Appl. No.: 14/131,954

(22) PCT Filed: Jul. 12, 2012
(86) PCT No.: PCT/EP2012/063729

§ 371 Date: Jul. 24, 2014

(30) Foreign Application Priority Data
Jul. 13, 2011 (DE) 10 2011 107 197.4
Aug. 19, 2011 (DE) 10 2011 111 044.9

(51) Int. Cl. C07K 1/18

(52) U.S. Cl. CPC C07K 1/18
USPC 530/305; 530/388.1

(57) ABSTRACT

The present invention relates to a crosslinked sulphonated polymer or a crosslinked sulphonated polymer coated with a crosslinked polymer containing amino groups for use as an ion exchanger material of high salt tolerance for separating off macromolecules from a solution which originates from a biological source.

The present invention relates to a crosslinked sulphonated polymer or a crosslinked sulphonated polymer coated with a crosslinked polymer containing amino groups for use as an ion exchanger material of high salt tolerance for separating off macromolecules from a solution which originates from a biological source.

The Coulomb interaction of ion exchanger resins is the interaction used the most in chromatographic purification processes. In ion exchanger resins, ionic groups, such as strong acids (for example sulphonic acid), strong bases (for example quaternary amines), weak acids (e.g. carboxylic acids) and weak bases (e.g. primary or tertiary amines) are preferably applied covalently as groups to a rigid matrix material. These ionic groups interact with complementary functional groups of the molecules to be purified, which are thus bonded to the ion exchanger resin. The elution of the target molecules bonded by ionic interaction is conventionally achieved by an increase in the salt concentration in the eluting agent, so that the target molecule is replaced by one or more corresponding salt ion(s). Relatively low salt concentrations of less than 150 mmol/l are conventionally sufficient to break the Coulomb interaction and to elute the target molecule.

Depending on the origin of the mixture from which the target molecule is to be separated, the salt concentration can already be higher than concentrations which can conventionally be used for the elution. This usually has the disadvantage that the target molecules do not bond to the ion exchanger resin in the presence of the high salt concentration. In particular, in solutions which are obtained from biological sources, such as fermentation liquids, body fluids or plant extracts, the conductivity (electrical conductivity; a parameter corresponding to the salt concentration) is usually too high for the direct use of ion exchanger chromatography. An undesirable dilution step is therefore often necessary in order to reduce the conductivity of the mixture (reduction in the salt concentration).

There are numerous known and available ion exchanger resins which are capable of bonding substances at a relatively high salt concentration. Nevertheless, all the ion exchanger resins known to date are no longer capable of bonding biological macromolecules, such as, for example, insulin, with an adequate loading capacity at concentrations of more than 250 mmol/l of sodium chloride. Sodium chloride is to be mentioned here only by way of example; in principle, however, other salts may also be present in this molar amount. Furthermore, the ion exchanger resins used which are known to date are not stable over the entire pH range of from pH 1 to 14 and therefore cannot be employed universally.

It was therefore an object of the present invention to provide a process for separating off macromolecules, in which the macromolecules can be separated off directly from a solution which originates from biological sources. It is furthermore desirable for the ion exchanger material used to be stable over a pH range of from 1 to 14. Due to its high salt tolerance, the ion exchanger material should render it possible that no additional dilution step has to be carried out in order to reduce the salt concentration. Such a process would have the advantage that the costs for solvents for the additional dilution step and for the processing of waste substances in the purification of mixtures comprising salt could be reduced.

To achieve the object mentioned, the present application provides the use of a crosslinked sulphonated polymer for separating off a macromolecule from a solution which originates from a biological source, wherein the crosslinked sulphonated polymer contains a sulphonated aromatic unit, which is substituted by an aliphatic radical or unsubstituted, bonded to its basic framework.

In other words, the present application relates to a process for separating off a macromolecule from a solution which originates from a biological source using a crosslinked sulphonated polymer containing a sulphonated aromatic unit, which is substituted by an aliphatic radical or unsubstituted, bonded to its basic framework.

According to the invention, macromolecules are understood as meaning molecules which have a molecular weight of greater than or equal to 10,000 g/mol. The macromolecule is particularly preferably a biomolecule, such as, for example, peptides and proteins, DNA, RNA, polysaccharides and lipopolysaccharides, such as, for example, endotoxins.

The term “separating off” is to be understood as meaning both the isolation/purification of a target molecule from the solution and the removal of undesirable macromolecules from the solution, so that the target molecule remains in the purified solution.

The basic framework of the crosslinked sulphonated polymer can be any known polymeric basic framework which is made of hydrocarbon-containing recurring units.

The basic framework of the polymer is understood as meaning the main chain of the polymer, to which sub-groups, such as the sulphonated aromatic unit, can be bonded in the form of side chains. In addition to the sulphonated aromatic unit, the polymer can also contain still further side chains, which are not to be considered as the basic framework but—as stated—are to be counted as side chains. In other words, the basic framework includes all atoms which build up the main chain of the polymer and are linked with at least two further at least bivalent atoms of the main chain. Single-bonding atoms, such as hydrogen atoms, which bond to the atoms mentioned are likewise counted as atoms of the basic framework. In the case where the crosslinked sulphonated polymer is crosslinked polystyrene, the linked vinyl units would be the basic framework and the sulphonated phenyl groups would be the side chains.

Hydrocarbon-containing recurring units are understood as meaning all conceivable compounds which are built up predominantly from carbon and hydrogen, but may also comprise hetero atoms. The linking of the recurring units to a polymer can be effected by any known polymerization process. Free radical, cationic or anionic olefin polymerization is particularly preferred according to the invention. The basic framework is particularly preferably a polyvinyl framework. The basic framework is preferably a crosslinked basic framework, so that a crosslinked polymer is formed. In the case of a polyvinyl framework in particular, the crosslinking arises by copolymerization of a monomer containing vinyl groups with a monomer which contains two vinyl groups.

However, it is in principle also conceivable for a polymer which has a linear basic framework first to be prepared. The subsequent crosslinking can then be carried out by reaction of functional groups in the side chain with a crosslinking reagent.

The crosslinked sulphonated polymer used according to the invention preferably contains sulphonic acid groups in the side chain. The side chains in the crosslinked sulphonated polymer according to the invention are sulphonated aromatic units, as described in detail below. The sulphonated aromatic units are preferably bonded to the basic framework by a covalent single bond. The sulphonated aromatic units can furthermore be substituted by an aliphatic radical. It is particularly preferable for the sulphonated aromatic units to be bonded directly to an atom of the basic framework by a covalent single bond.

In the present invention an aromatic unit is understood as meaning a mono- or polycyclic aromatic ring system which is substituted by an aliphatic radical or unsubstituted. In the context of this invention, an aromatic ring system is understood as meaning preferably an aromatic ring system having 6 to 60 carbon atoms, preferably 6 to 30, particularly preferably 6 to 10 carbon atoms. These aromatic ring systems can be monocyclic or polycyclic, i.e. they can have one ring (e.g. phenyl) or two or more rings, which can also be fused (e.g. naphthyl) or covalently linked (e.g. biphenyl), or comprise a combination of fused and linked rings.

Preferred aromatic ring systems are, for example, phenyl, biphenyl, triphenyl, naphthyl, anthracyl, binaphthyl, phenanthryl, dihydrophenanthryl, pyrene, dihydropyrene, crysene, perylene, tetracene, pentacene, benzpyrene, fluorene and indene. Particularly preferred aromatic ring systems are phenyl, biphenyl or naphthyl, particularly preferably phenyl.

As already mentioned, the aromatic ring systems can be substituted by an aliphatic group. It is conceivable here for the aromatic ring system to be substituted not only by one but by two or more aliphatic groups. An aliphatic radical is preferably a hydrocarbon radical having 1 to 20, or 1 to 10 carbon atoms. Aliphatic hydrocarbon radicals according to the invention are preferably linear or branched or cyclic alkyl groups in which one or more hydrogen atoms can also be replaced by fluorine. Examples of the aliphatic hydrocarbon radicals having 1 to 20 hydrocarbon atoms include the following: methyl, ethyl, n-propyl, iso-propyl, n-butyl, iso-butyl, sec-butyl(1-methylpropyl), tert-butyl, iso-pentyl, n-pentyl, tert-pentyl(1,2-dimethylpropyl), 1,2-dimethylpropyl, 2,2-dimethylpropyl(neopentyl), 1-ethylpropyl, 2-methylbutyl, n-hexyl, iso-hexyl, 1,2-dimethylbutyl, 1-ethyl-1-methylpropyl, 2-methylbutyl, 1-ethyl-2-methylpropyl, 1,1,2-trimethylpropyl, 1,2,2-trimethylpropyl, 1-ethylbutyl, 1-methylbutyl, 1,1-dimethylbutyl, 2,2-dimethylbutyl, 1,3-dimethylbutyl, 2,3-dimethylbutyl, 3,3-dimethylbutyl, 2-ethylbutyl, 1-methylpentyl, 2-methylpentyl, 3-methylpentyl, cyclopentyl, cyclohexyl, cycloheptyl, cyclooctyl, 2-ethylhexyl, trifluoromethyl, pentafluoroethyl and 2,2,2-trifluoroethyl. Methyl or ethyl is particularly preferred as the aliphatic hydrocarbon radical.

It is exceptionally preferable for the sulphonated aromatic unit of the crosslinked sulphonated polymer which is substituted by an aliphatic radical or unsubstituted to be a phenylsulphonic acid group or a derivative thereof. In the case of the derivative of the phenylsulphonic acid group, this means derivatives which are substituted by an aliphatic radical. In this case a sulphonic acid group on the phenyl radical is preferably in the para position relative to the position on the phenyl ring which bonds to the basic framework. The aliphatic radical here is preferably a methyl or ethyl group, which is in the ortho and/or meta position on the phenyl group relative to the position on the phenyl ring which bonds to the basic framework.

However, it is particularly preferable for the sulphonated aromatic unit not to be substituted. A sulphonated crosslinked polystyrene in particular is possible here. The crosslinking of the sulphonated polystyrene is preferably carried out by copolymerization of styrene with divinylbenzene, followed by sulphonation of the phenyl groups. However, any other crosslinking agents containing two vinyl groups are conceivable here for the preparation of a crosslinked copolymer.

According to the invention, the degree of crosslinking of the crosslinked sulphonated polymer is preferably 0.5 to 50%, particularly preferably 5 to 45% and most preferably 10 to 35%. In the present invention indication of the degree of crosslinking in percent is understood as meaning the percentage molar content of the compound containing two vinyl groups which is employed relative to the total number of monomer units to be polymerized.

The degree of sulphonation of the crosslinked sulphonated polymer is preferably 1 to 80%, more preferably 3 to 60% and most preferably 5 to 40%. Indication of the degree of sulphonation in percent relates to the number of moles of sulphonic acid groups in relation to all the monomer units containing a sulphonatable group which are employed for the polymerization. Monomer units containing a sulphonatable group which are employed for the polymerization are understood as meaning all monomer units which contain the sulphonated aromatic unit and also all the monomer units which contain a sulphonatable group, preferably an aromatic unit, and optionally all compounds which cause the crosslinking if these contain a sulphonatable or sulphonated group. If sulphonated polystyrene/divinylbenzene copolymer is used as the crosslinked sulphonated polymer, the degree of sulphonation in percent relates to the number of sulphonic acid groups in relation to all the phenyl or phenylene groups contained in the polymer.

The crosslinked sulphonated polymer employed in the process according to the invention or in the use according to the invention is preferably present in the form of regularly or irregularly shaped resin particles. In the present invention the term “regularly shaped” is understood as meaning shapes which can be represented by symmetry operations, such as surface mirror image, point mirror image or axes of rotation or combinations thereof. The spherical shape is particularly preferably to be mentioned here. The term “spherical” is understood as meaning not only purely symmetrical spheres, but also shapes which deviate therefrom, such as, for example, ellipses. However, two spherical bodies joined to one another to give dumbbells are also to be included here. An irregular shape is understood as meaning any broken shape which has no symmetry. The resin particles preferably have a mean average diameter of from 1 to 1,000 μm, more preferably from 5 to 100 μm and particularly preferably from 10 to 50 μm.

The crosslinked sulphonated polymer employed in the process according to the invention or in the use according to the invention preferably has pores in which the actual interaction with the substances to be separated takes place. It is thus preferably a porous polymer material. These pores preferably have an average diameter of from 6 to 400 nm, particularly preferably from 30 to 100 nm. The pore diameter is determined by an inverse size exclusion chromatography. In this, the phase material to be investigated is packed into a chromatography column and a series of polymer size standards is injected. From the course of the curve in the plot of the logarithm of the molecular weight of the particular standard against the elution volume, the distribution of the pore diameters and therefore the average pore diameter can be determined by methods known from the literature.

It is furthermore preferable for the crosslinked sulphonated polymer to have a pore volume in the range of from 1 to 3 ml/g. The pore volume is determined by measurement of the water uptake capacity. The solvent for which the pore volume is to be determined (different solvents can display different results because of different wettability) is added to the phase material, of which the weight in the dry state has been determined. Water is used as the solvent for the purposes of the present invention. Excess solvent is filtered off and the phase material is freed from further solvent in the interparticle volume in a centrifuge. The material is then weighed again. Only the pores should still be filled with the solvent. The pore volume can be calculated via the difference in weight between the filled and empty pores and the density of the solvent.

The crosslinked sulphonated polymer employed in the process according to the invention or in the use according to the invention has the advantage that, in addition to the lipophilic basic framework with the aromatic units in the side chain, it also contains ionizable groups, such as sulphonic acid groups. In this manner it is suitable for interaction with the macromolecule by both ionic interactions and lipophilic interactions. The sulphonic acid groups here preferably serve as anionic —SO₃⁻ groups which are capable of undergoing ionic interactions with cations of the macromolecule. Furthermore, macromolecules from biological sources, such as, for example, proteins, DNA or RNA, also have lipophilic regions which can interact with the aromatic units of the crosslinked sulphonated polymer as a lipophilic matrix. In this manner it is possible to employ solutions which originate from a biological source and which can comprise a very high salt content of up to 1 mol/l of salt without elution of the macromolecules from the ion exchanger material occurring.

The crosslinked sulphonated polymer used according to the invention is preferably employed for the isolation or purification of macromolecules containing cationic groups. The macromolecule is preferably a biological macromolecule. The biological macromolecule is preferably a peptide. The peptide is very particularly preferably insulin. In other words, the present invention thus preferably relates to a use of the crosslinked sulphonated polymer for the purification or isolation of insulin from a solution which originates from a biological source.

The preparation of the crosslinked sulphonated polymer is preferably carried out by sulphonation of an already crosslinked polymer by employing sulphuric acid and similar materials, such as is known, for example, for the preparation of sulphonated crosslinked polystyrene from the British patents GB 1116800 and GB 1483587. The preparation of crosslinked polymers is state of the art and can be carried out by any person skilled in the art in the field of polymer chemistry without an inventive step.

Particularly preferably, however, the sulphonation is carried out as follows: depending on the desired degree of sulphonation, for example, a polystyrene/divinylbenzene polymer is stirred in a mixture of sulphuric acid and water having a water content of from 2 to 15% at temperatures of from 20° C. to 80° C. for 1 to 6 hours. The increase in the sulphuric acid content, the temperature and the reaction time each in itself leads to an increase in the degree of sulphonation. By adjusting all three parameters, the desired degree of sulphonation can be achieved relatively precisely. After the reaction the polymer is rinsed with dilute sulphuric acid and water.

According to the invention it is also preferable for the crosslinked sulphonated polymer to be coated with a crosslinked polymer containing amino groups.

The basic framework of the crosslinked polymer containing amino groups is preferably the same as mentioned above for the crosslinked sulphonated polymer. The basic framework is thus also particularly preferably a polyvinyl framework here. On this polyvinyl framework, amino groups are preferably linked directly to atoms of the basic framework by covalent single bonds.

According to the invention, amino groups are understood as meaning primary, secondary, tertiary or quaternary amino groups, as well as also amidine or guanidine groups. The crosslinked polymer containing the amino groups, however, is particularly preferably a crosslinked polyvinylamine.

The crosslinking of the crosslinked polymer containing amino groups is preferably carried out by reacting a linear polymer which contains primary or secondary amino groups with a crosslinking reagent which can form covalent bonds with the amino groups on two ends. In principle any conceivable crosslinking reagent can be employed for this. According to the invention, however, crosslinking reagents in which all the amino groups used for the crosslinking are still present in the form of an amino group after the crosslinking are particularly preferably employed. In this manner it is ensured that the amino groups, by protonation/alkylation, are still capable of functioning as cationic ion exchanger groups. This leads to a high density of ion exchanger groups on the otherwise lipophilic matrix. After the crosslinking the previously primary or secondary amino groups are then present as secondary or tertiary amino groups.

In order to impart a positive charge to the amino groups, these can be protonated. As an alternative to this, however, primary, secondary or tertiary amino groups can also be converted into quaternary ammonium ions by tri-, bi- or monoalkylation with an alkylating reagent.

The degree of crosslinking of the crosslinked polymer containing amino groups is preferably in the range of from 5 to 80%, particularly preferably in the range of from 6 to 60% and most preferably in the range of from 10 to 40%. The percentage figure here relates to the number of amino groups used for the crosslinking in relation to all the amino groups of the non-crosslinked polymer.

It is particularly preferable for the weight ratio of the crosslinked polymer containing amino groups to the crosslinked sulphonated polymer to be in the range of from 0.05 to 0.3, particularly preferably from 0.08 to 0.25 and most preferably from 0.11 to 0.20.

The crosslinked polymer containing amino groups is preferably present in the form of a layer/coating on the crosslinked sulphonated polymer. The crosslinked sulphonated polymer is preferably employed here in the form of resin particles and is coated with the non-crosslinked polymer containing amino groups and is then crosslinked with the crosslinking agent. In this manner a high concentration of amino groups can be realized on the surface, without the lipophilic properties of the matrix being lost completely by this process. An ion exchanger resin which, by protonation/alkylation of the amino groups, is capable of interacting with anionic groups of the macromolecule is thus provided. In addition, the lipophilic matrix can also undergo lipophilic interactions with the macromolecule.

The crosslinked polymer containing amino groups which is present on the surface of the sulphonated polymer is preferably deposited in the pores of the resin particles of the sulphonated polymer, i.e. it is preferably present in the pores of the sulphonated polymer.

The crosslinked polymer containing amino groups preferably has an average molecular weight in the range of from 20,000 to 50,000 g/mol, more preferably 30,000 to 46,000 g/mol.

Particularly preferably, macromolecules such as DNA or RNA are removed from the solutions by this cationic ion exchanger resin, so that the solution is purified from this, and desired target molecules without DNA or RNA can be isolated from the solution.

Equally preferably, macromolecules such as endotoxins are removed from the solutions by the ion exchanger resin (anion exchanger) according to the invention, so that these initially remain bonded to the ion exchanger resin and the solution is present in a form mostly free from the endotoxins. Either the original solution can be freed from the endotoxins in this way and used further, or the endotoxins can be obtained by elution from the ion exchanger resin with a suitable solution. Endotoxins are understood as meaning a class of biochemical substances. They are decomposition products of bacteria which can trigger numerous physiological reactions in humans. Endotoxins are a constituent of the outer cell membrane (OM=outer membrane) of Gram-negative bacteria or blue algae. Chemically they are lipopolysaccharides (LPS), which are built up from a hydrophilic polysaccharide content and a lipophilic lipid content. In contrast to the bacteria from which they originate, endotoxins are very heat-stable and withstand even sterilization. Currently the most sensitive method for measurement of endotoxins functions via activation of the coagulation cascade in the lysate of amoebocytes which have been isolated from horseshoe crabs (Limulus polyphemus). This test is known generally as the LAL test.

As already mentioned, the macromolecules according to the invention originate from biological sources. The macromolecules here preferably have a molecular weight in the range of from 1,000 to 0.2 kDa, more preferably 500 to 1 kDa and most preferably from 300 to 5 kDa.

A solution which originates from a biological source is understood as meaning solutions which are obtained, for example, by fermentation or fermentation processes, body fluids or plant extracts, which preferably have an ionic conductivity in the range of from 0.1 mS/cm to 120 mS/cm, more preferably in the range of from 1 to 60 mS/cm and most preferably from 10 to 20 mS/cm. These solutions are preferably aqueous solutions. They preferably have a salt content of up to 1.2 mol/l. Particularly preferably, their salt content is in the range of from 0.01 to 1.2 mol/l, more preferably in the range of from 0.05 to 1.0 mol/l and most preferably in the range of from 0.25 to 0.6 mol/1. In the present invention a salt is understood as meaning any salt, such as inorganic and organic salts, which are preferably present in biological liquids. These solutions are understood here as meaning not only solutions which are obtained and used directly from the biological sources, but also solutions which have already been processed in some manner. “Processed” is understood as meaning that the solutions have been pretreated in some manner, for example changing of the pH or separating off of substances before the use according to the invention.

The ionic conductivity is determined according to the invention with a conductivity meter from Greisinger, type GMH 3430.

With the crosslinked sulphonated polymers used according to the invention or the crosslinked sulphonated polymers coated with a layer of a crosslinked polymer containing amino groups, biological macromolecules can thus be bonded from solutions having an extremely high salt content, without the solutions having to be diluted beforehand by additional dilution steps or dialyses. In this manner the present invention provides an inexpensive process/an inexpensive use for the purification of biological macromolecules, preferably insulin, monoclonal antibodies, DNA or RNA. In addition, the ion exchanger materials used have the advantage that they can be employed in the entire pH range of from 1 to 14, such as occurs in liquids originating from biological sources.

The present invention furthermore also relates to the further embodiments:
(i) Process for separating off a macromolecule from a solution which originates from a biological source using a crosslinked sulphonated polymer containing a sulphonated aromatic unit, which is substituted by an aliphatic radical or unsubstituted, bonded to its basic framework.
(ii) Process according to embodiment (i), wherein the basic framework is a crosslinked polyvinyl framework.
(iii) Process according to embodiment (i) or (ii), wherein the aromatic unit is a phenylsulphonic acid group.
(iv) Process according to one of embodiments (i) to (iii), wherein the crosslinked sulphonated polymer is a sulphonated polystyrene/divinylbenzene copolymer.
(v) Process according to one of embodiments (i) to (iv), wherein the degree of crosslinking of the crosslinked sulphonated polymer is 0.5 to 50%.
(vi) Process according to one of embodiments (i) to (v), wherein the degree of sulphonation is 1 to 80%, based on the number of moles of sulphonic acid groups in relation to all the sulphonatable monomer units employed for the polymerization.
(vii) Process according to one of embodiments (i) to (vi), wherein the crosslinked sulphonated polymer is present in the form of resin particles.
(viii) Process according to embodiment (vii), wherein the resin particles have a mean average diameter of from 1 to 1,000 μm.
(ix) Process according to embodiment (vii) or (viii), wherein the resin particles have pores having an average diameter in the range of from 10 to 400 nm.
(x) Process according to one of embodiments (i) to (ix), wherein the macromolecule is a peptide.
(xi) Process according to embodiment (x), wherein the peptide is insulin.
(xii) Process according to one of embodiments (i) to (ix), wherein the crosslinked sulphonated polymer is coated with a crosslinked polymer containing amino groups.
(xiii) Process according to embodiment (xii), wherein the degree of crosslinking of the polymer containing amino groups is in the range of from 5 to 80%.
(xiv) Process according to embodiment (xii) or (xiii), wherein the crosslinked polymer containing amino groups is crosslinked polyvinylamine.
(xv) Process according to one of embodiments (xii) to (xiv), wherein all the amino groups used for the crosslinking are present in the form of an amine after the crosslinking.
(xvi) Process according to one of embodiments (xii) to (xv), wherein the weight ratio of the crosslinked sulphonated polymer to the crosslinked polymer containing amino groups is in the range of from 3 to 20.
(xvii) Process according to one of embodiments (xii) to (xvi), wherein the macromolecule is an endotoxin, DNA or RNA.

The invention is to be explained in the following with the aid of figures and examples which, however, are not to be understood as limiting the scope of protection.

### FIGURES

FIG. 1: Comparison of an ion exchanger used according to the invention with the uses, not according to the invention, of two ion exchangers according to the state of the art by measurement of the loading capacity with insulin as a function of the salt concentration.

FIG. 2: Plot of the extinction of the eluate against time after passage of a fermentation solution through an anion exchanger material used according to the invention.

FIG. 3: Comparison of an ion exchanger used according to the invention with the uses, not according to the invention, of two ion exchangers according to the state of the art by measurement of the loading capacity with DNA as a function of the salt concentration.

### EXAMPLES

### Example 1

### Preparation of a Cation Exchanger Resin Based on a Crosslinked Sulphonated Polymer

Aim of the set-up: Sulphonation of the polystyrene support Amberchrom XT 30 (commercially obtainable from The Dow Chemical Company, formerly Rohm & Haas) at 20° C.

165 ml of conc. H₂SO₄ were introduced into a temperature-controllable 250 ml reactor. 30.0 g of the support material were added to the sulphuric acid and the weighing bottle was rinsed three times with 20 ml of conc. sulphuric acid each time. After the addition of the support material, the suspension was stirred, and temperature-controlled at 20° C. After a reaction time of 2 h the suspension was drained off from the reactor and distributed over two 150 ml syringes. The sulphuric acid was filtered off with suction and the phase was rinsed successively with 200 ml of dilute (62% strength) sulphuric acid, 125 ml of water, 175 ml of methanol, 125 ml of water and finally with 175 ml of methanol. The phase was sucked dry and then dried at 50° C. in vacuo.

The determination of the sulphonic acid groups is carried out in an HPLC column by loading with ammonium acetate, subsequent elution of the ammonium bonded and detection via indophenol blue. A sulphonic acid content of 375 μmol/ml resulted. This corresponds to a degree of sulphonation of approximately 13%. The particle size is on average 30 μm. The particles are spherical with an average pore diameter of 22 nm and an average pore volume of 1.25 ml/g.

### Example 2

### Preparation of an Anion Exchanger Based on a Crosslinked Sulphonated Polymer Coated with a Crosslinked Polymer Containing Amino Groups

Amberchrom CG1000S from Rohm & Haas is used as the basis for the ion exchanger material. This is sulphonated, as explained in Example 1, with 98% strength sulphuric acid at 80° C. for 3 hours. Particles having a mean average size of 30 μm and an average pore diameter of from 22 to 25 nm are obtained by this procedure. The water uptake capacity or the pore volume of the resulting sulphonated polystyrene is determined by weighing the dried, sulphonated polystyrene, adding the same volume of water and then centrifuging off excess water. The water in the pores remains in its position by this procedure. After weighing again, the pore volume can be determined as about 1.2 to 1.3 ml/g from the difference in weight from the dry polystyrene.

For coating the polystyrene, an aqueous polyvinylamine solution which comprises polyvinylamine having an average molecular weight of 35,000 g/mol is prepared. The pH value is adjusted to 9.5. The amount of polyvinylamine here is 15% of the polystyrene to be coated, and the volume of the solution is 95% of the pore volume determined for the polystyrene. The polyvinylamine solution is introduced into a firmly closed PE bottle together with the polystyrene and the mixture is shaken on a screen shaker at a high frequency for 6 hours. Adequate thorough mixing must be ensured here. After the procedure, the polyvinylamine solution has worked itself into the pores of the polystyrene. The polystyrene is then dried to constant weight at 50° C. in a vacuum drying cabinet. For crosslinking of the polyvinylamine, the coated polystyrene is taken up in three times the volume of isopropanol, and 5% of diethylene glycol diglycidyl ether, based on the number of amino groups of the polyvinylamine, is added. The reaction mixture is stirred in the reactor at 55° C. for six hours. It is then transferred to a glass suction filter and rinsed with 2 bed volumes of isopropanol, 3 bed volumes of 0.5 M TFA solution, 2 bed volumes of water, 4 bed volumes of 1 M sodium hydroxide solution and finally 8 bed volumes of water.

### Example 3

### Purification of Insulin by the Cation Exchanger Prepared in Example 1

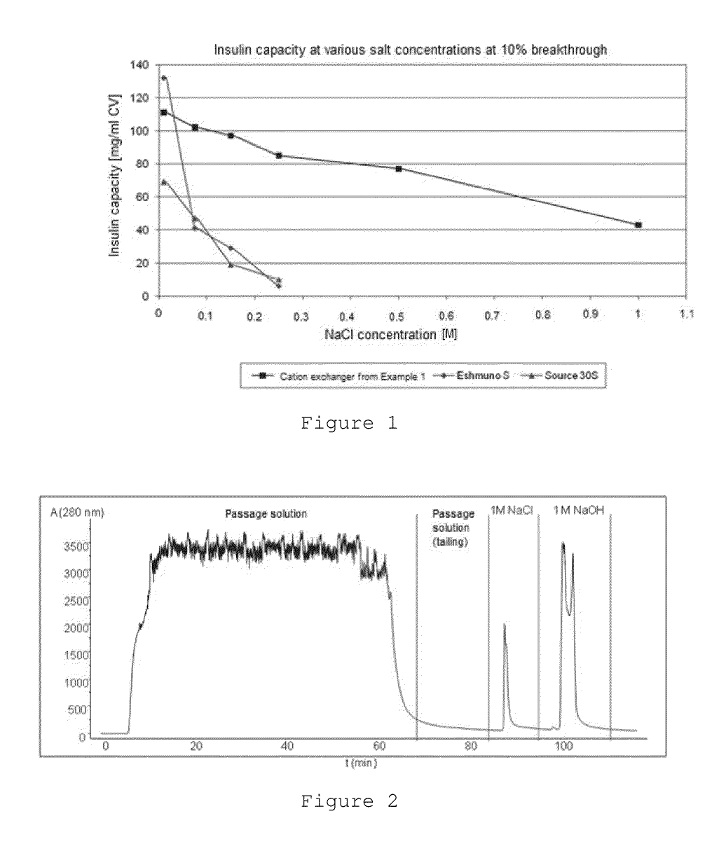

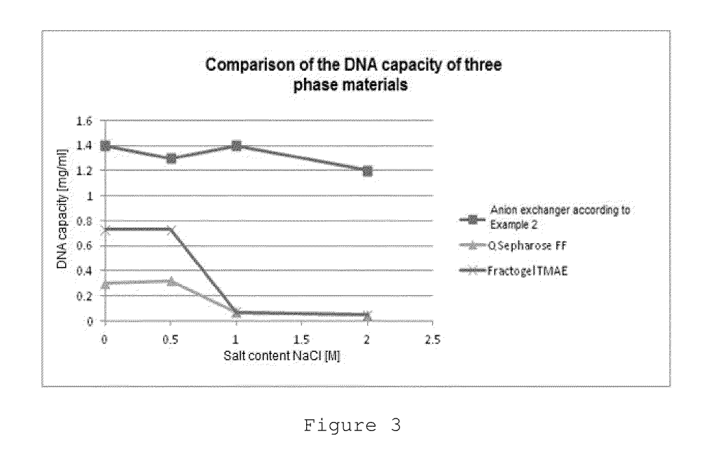

The determination of the loading capacity with insulin of the salt-tolerant ion exchanger prepared in Example 1 is carried out with a solution of 10 mg/ml of insulin in 30% isopropanol with 50 mM lactic acid at pH 3.5 and various concentrations of NaCl. The loading capacity was determined at 10% breakthrough and compared with two competing materials. The results are shown in FIG. 1. The comparison materials used were the commercially obtainable ion exchanger materials “Eshumo S” from Merck (polyvinyl ether, ionic capacity 50-100 μmol/ml, particle size 75-95 μm) and “Source 30S” from GE Healthcare (polystyrene/divinylbenzene, particle size 30 μm).

While at an NaCl content of 250 mM in the mobile phase the comparison materials show only a very low capacity, which is no longer measurable at higher salt contents, the ion exchanger used according to the invention still shows a significant capacity up to 1 M NaCl. This can be clearly seen from FIG. 1.

### Example 4

### Separating Off of DNA by Employing the Anion Exchanger Resin Prepared in Example 2

The first step in the process of purification of monoclonal antibodies from fermentation solutions is the depletion of the DNA contained therein. This functions by “filtration” of the fermentation solution over a phase of the anion exchanger prepared in Example 2. In this step the DNA bonds to the phase, and the fermentation solution passing through quantitatively is almost freed from the DNA in this way. For this, the anion exchanger prepared in Example 2 is packed into a 270×10 mm column with a bed volume of 21.2 ml and equilibrated with first 500 mM NaKPO₄ pH 7.0 and then with 50 mM NaKPO₄ pH 7.0. The fermentation solution is filtered over a 0.45 μm filter and freed from precipitates. 300 ml of the fermentation solution are introduced on to the column via an external pump. The passage solution, the eluate with 1 M NaCl pH 6.5 and the rinsing step with 1 M NaOH are collected.

The part in FIG. 2 called passage solution comprises almost exclusively the monoclonal antibody and no DNA. Elution of the DNA takes place, however, only by the application of NaOH.

The content of DNA in the passage solution and in the fermentation solution is determined with a PicoGreen assay in accordance with the manufacturer's instructions.

TABLE 1

| dsDNA | |
|---|---|
| fermentation solution | dsDNA |
| (filtered) | passage solution |
| 4,767 μg | 30 μg |
| 100% | 0.7% |
| 9,046 ppm | 57 ppm |

It can be seen from Table 1 that 99.3% of the DNA could be removed in the filtration over the phase material.

The bonded DNA is not eluted in the 1 M NaCl step, but only by rinsing with 1 M NaOH, since the amino groups of the phase are deprotonated here and bonding to the DNA is no longer present.

As an alternative to the anion exchanger prepared in Example 2, the commercially obtainable materials Q Sepharose FF from Amersham Biosciences and Fractogel TMAE from Merck were also used as separating agents as in Example 4. In the determination of the static capacity at various salt contents compared with Q Sepharose FF and Fractogel TMAE, a higher loading capacity of the ion exchanger developed results even at high salt contents.

### Example 5

### Separating Off of Endotoxins from Fermentation Solutions by Employing the Anion Exchanger Resin Prepared in Example 2

A fermentation solution which comprises endotoxins is “filtered” over a phase of the anion exchanger prepared in Example 2. In this step the endotoxins bond to the phase, and the fermentation solution passing through quantitatively is almost freed from the endotoxins in this way. For this, the anion exchanger prepared in Example 2 is packed into a 270×10 mm column with a bed volume of 21.2 ml. The fermentation solution is filtered over a 0.45 μm filter and freed from precipitates. 300 ml of the fermentation solution are introduced on to the column via an external pump.

The passage solution from the column comprises at least 90% less endotoxins than the fermentation solution. The LAL test was used to detect the endotoxin content. In this manner the fermentation solution could be freed from a large proportion of the endotoxins. The endotoxins were then washed from the ion exchanger with a suitable eluate.

1-16. (canceled)

17. A method of chromatographic purification for separating off a macromolecule from a solution which originates from a biological source by use of a crosslinked sulphonated polymer, wherein the crosslinked sulphonated polymer contains a sulphonated aromatic unit, which is present in a form substituted by an aliphatic radical or an unsubstituted form, bonded to a basic framework.

18. The method of claim 17, wherein the basic framework is a crosslinked polyvinyl framework.

19. The method of claim 17, wherein the sulphonated aromatic unit is a phenylsulphonic acid group.

20. The method of claim 17, wherein the crosslinked sulphonated polymer is a sulphonated polystyrene/divinylbenzene copolymer.

21. The method of claim 17, wherein the degree of crosslinking of the crosslinked sulphonated polymer is 0.5 to 50%.

22. The method of claim 17, wherein the degree of sulphonation is 1 to 80%, based on the number of moles of sulphonic acid groups in relation to all the sulphonatable monomer units employed for the polymerization.

23. The method of claim 17, wherein the crosslinked sulphonated polymer is present in the form of resin particles.

24. The method of claim 23, wherein the resin particles have a mean average diameter of from 1 to 1,000 μm.

25. The method of claim 23, wherein the resin particles have pores having an average diameter in the range of from 10 to 400 nm.

26. The method of claim 17, wherein the macromolecule is a peptide.

27. The method of claim 26, herein the peptide is insulin.

28. The method of claim 17, wherein the crosslinked sulphonated polymer is coated with a crosslinked polymer containing amino groups.

29. The method of claim 28, wherein the degree of crosslinking of the crosslinked polymer containing amino groups is in the range of from 5 to 80%.

30. The method of claim 28, wherein the crosslinked polymer containing amino groups is crosslinked polyvinylamine.

31. The method of claim 28, wherein all the amino groups used for the crosslinking are present in the form of an amine after the crosslinking.

32. The method of claim 28, wherein the macromolecule is an endotoxin, DNA or RNA.
